(19) United States
(12) Patent Application Publication
(10) Pub. No.: US 2005/0197540 A1
(43) Pub. Date: Sep. 8, 2005

(54) Dermal diagnostic system including an active transponder

(75) Inventor: Rainer K. Liedtke, Gruenwald (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BIONICS PHARMA GmbH, Gruenwald (DE)

(21) Appl. No.: 10/994,236

(22) Filed: Nov. 23, 2004

(30) Foreign Application Priority Data
Nov. 24, 2003 (DE) 103 54 833.5

(51) Int. Cl. A61B 5/00; A61B 5/04; G08B 13/14

(52) U.S. Cl. 600/300; 128/903; 600/549; 600/546; 600/509; 600/306

(57) ABSTRACT

A dermal diagnostic system including an active transponder for an improved mobile and non-invasive detection, transmission, and processing of medically relevant biological base data. The active transponder works in conjunction with at least one sensor arranged in or at an adhesive flexible carrier such that the sensor is provided in adequate measuring contact with the skin surface or subjacent layers and transmit their data to the transponder for external transmission. The dermal system is moreover adapted to be technically connected directly with secondary communication networks. Such a system offers improved medical-diagnostic possibilities.

### BACKGROUND OF THE INVENTION

1. Field of the Invention

The present invention relates to a dermal diagnostic system including an active transponder, especially for an improved mobile determinaion, transmission, and processing of biological data.

Such a technical device can be classified as a dermal diagnostic system that is applied to the skin and by which information about the health condition of the body can be obtained from the skin surface, from dermal layers or subjacent layers. This includes information about biophysical body parameters or the content in quality or quantity of endogenous or exogenous substances.

2. Background of the Invention

Biophysical parameters such as temperature, pulse frequency, and blood pressure are clinical base parameters for the routine determination of a physical condition. Accordingly, they have a considerable medical significance. The same is applicable to the determination of clinic chemical base parameters, for instance to the measurement of blood sugar with diabetics or of electrolytes. At present these parameters are largely detected by measuring instruments in which measured values are both determined and evaluated directly at the place of measurement. These instruments usually have large dimensions and moreover require numerous cable connections that, on the one hand, restrict the mobility of a user or patient and, on the other hand, bind the use of these instruments to one place for the measuring period. The numerous instruments often required for measurements of this type are evident, for instance, in the monitoring of the intensive care. Moreover, considerable operational efforts as well as risks due to additional fault sources result from cable connections.

It is known that, currently, there are various methods of wireless data transmission, especially those in which measuring instruments are connected to evaluating instruments via optical waves, for example infrared (IR) or radio waves. Such background systems are complex and expensive, however, and thus the widespread use thereof is limited.

It is known that in certain industrial applications an innovative technology works with transponders on the basis of a radio frequency identification technology (RFID). The term transponder is a combination of the terms transmitter and responder. Transponders thus are basically wireless communication devices that receive and transmit wave signals of particular frequencies. This general definition of the transponder thus includes waves of all frequencies and therefore is not restricted to radio frequencies, but also includes optical waves such as infrared. However, from the signal frequencies used technically different possibilities result, and herefrom different technical configurations of the transponders for the applications also result. At present transponders are used in satellite communication as well as in locating, identification, and navigation systems. More recent RFID transponders in identification systems are flat flexible carrier films including a simple memory chip containing particular predetermined information as well as an antenna of planar design. When a transponder passes a station including a transmitting and receiving unit, it transmits the identification information contained in the transponder upon a request by the station to the station. Other than in the known graphical bar codes, for instance, the transponders require no direct visual connection to their reading station and they can be read out through all non-magnetic substances.

In general, engineering the logistical chain of a technical product, for instance, can be traced largely completely from the manufacturer via the storage up to its sale by RFID tags or labels. Therefore, transponders are used now as novel technical ways for locating and identification for a production and distribution path. In the technical view, moreover, a distinction of RFID systems is made into passive and active transponders. Passive transponders have no separate energy supply. The energy for their operation, i.e. for the output of identification information stored in the transponder in advance, is supplied directly from the external transmitter, and therefore passive transponders have only very short ranges of approximately up to 50 cm. In contrast to that, active transponders, as they are called, also have their own energy source, preferably a battery. In this way they have by far longer ranges, at the moment of approximately up to 100 m. However, at present, active transponder systems are still largely in an experimental state, especially by reason of the considerable problems of integrating suitable battery systems as energy carriers in these structures having very small dimensions. Hereinafter the term active transponder refers to such systems in which an inherent energy supply is implemented.

### SUMMARY OF THE INVENTION

Apart from their present possibilities of locating and identifying products, the present inventor recognized that transponders are basically capable of performing further functions when they are combined with sensors. Depending on their intended use, further chips or sensors can be integrated, for instance those for measuring temperature, pressure, moisture, or a pH value. In the medical field transponders have not been used so far, and especially in the field of biological surface applications, in particular of the skin, as well as corresponding diagnostic purposes no developments including the use of active transponders have been known so far.

This is probably based on conceptual and biological-technical reasons, for instance on the lack of appropriate ways for arranging transponders on biological surfaces, the lack of mechanisms for appropriately arranging and positioning sensors for such biological purposes, as inter alia also the requirements of a mechanical flexibility of the material. Ultimately, another reason probably is the considerable problem of accommodating a suitable energy supply, because for such biomedical purposes longer ranges are required than in the industrial engineering sector so far.

Accordingly, one object underlying the present invention is to improve the mobile determination, transmission, and processing of biological data.

This object is achieved by using a dermal diagnostic system including an active transponder, wherein the system is composed as a multi-component system including an elastic or flexible carrier matrix in which an active transponder and sensor are integrated so that the sensor is in adequate measuring contact with the skin surface or with subjacent dermal layers, wherein the data detected by the sensor is supplied to the transponder for external transmission and the entire system is adhesively applied to the skin surface.

To improve and extend the practical use of the present invention, in a further embodiment of the present invention, additional electric, magnetic, optical, micromechanical, thermal, chemical, or chemical engineering components, or combinations thereof, are implemented in the diagnostic system.

To improve and extend the practical use of the present invention, in a further embodiment of the present invention the transponder and/or the sensor are reversibly arranged on the carrier, wherein the reversible arrangement can be performed through complementary devices on the carrier and the transponder chemically by reversible gluing, magnetically or mechanically by hook and loop (Velcro) fastener, snap fastener, or flange connections.

To improve and extend the practical use of the present invention, in another embodiment of the present invention functional parts of the transponder, especially of the energy supply thereof, are arranged separately in the carrier or in further carrier materials connected therewith.

To improve and extend the practical use of the present invention, in a further embodiment of the present invention the transponder also makes use of optical waves in lieu of radio waves or in combination with the use of radio waves as signals, for example infrared in particular.

To improve and extend the practical use of the present invention, in another embodiment of the present invention plural sensors are simultaneously contained in the system.

To improve and extend the practical use of the present invention, in a further embodiment of the present invention a sensor or plural sensors are integrated directly in the carrier matrix of the transponder.

To improve and extend the practical use of the present invention, a further embodiment of the present invention is used for checking the temperature, pulse frequency, blood pressure, moisture, and/or electric currents produced in muscles or action currents of the heart.

To improve and extend the practical use of the present invention, a further embodiment of the present invention is used for checking chemical parameters, especially blood sugar, electrolytes, and lactate.

To improve and extend the practical use of the present invention, a further embodiment of the present invention is provided with electric, magnetic, optical, or micromechanical components adapted to directly communicate with a mobile communication system, especially a mobile phone or portable computer, or to be connected to the mobile communication system.

To improve and extend the practical use of the present invention, a further embodiment of the present invention is used in the fields of human medicine, veterinary medicine, or biology.

### BRIEF DESCRIPTION OF THE DRAWINGS

A more complete appreciation of the present invention, and many of the attendant advantages thereof, will be more readily obtained as the same becomes better understood by reference to the following figures in which:

FIG. 1 shows a first embodiment of a dermal diagnostic system of the present invention;

FIG. 2 shows the first embodiment of FIG. 1 in an exploded view; and

FIG. 3 shows a modification of a first embodiment of the present invention.

### DESCRIPTION OF THE PREFERRED EMBODIMENTS

The advantages of the present invention result from the fact that for the first time there is a suitable possibility of determining biological data from biological surfaces, especially from the skin, for a technique that otherwise is only employed for the logistical locating and identification of industrial products via a new technical device including the modification and integration of active transponders in specific dermal systems suited for biological surfaces. Moreover this opens up numerous new medical applications.

General medical advantages of such novel dermal systems are that the application is technically non-invasive and harmless and permits a mobile, cable-free and close-meshed remote measurement of biologically very important body data. This is applicable, for instance, in the clinical sector for determining the course of temperature of operated patients for the purpose of prophylaxis against infections, or else the degree of moisture of wounds and wound dressings. Checks of this type are costly in the clinical routine, because a close-meshed control of the bedfast patients by the nursing staff is required. Although this is already possible at present, it can only be realized via more complex systems mostly including direct cabling, which means a considerably restricted mobility.

The dermal system of the present invention permits more close-meshed diagnostic of the progress, which moreover can be economically justified and has a substantial significance for medical prevention. Since important bio-data about the body condition can be quickly interrogated and transmitted without a restricted mobility, this enables a physician or the nursing staff to take counter-measures at an earlier time. Already regarding the clinical individual example of a temperature measurement, an efficient preventive check for possible risk factors is provided in this way, for instance of an upcoming infection or an inflammatory process, e.g. in surgery in the wake of operations, in pediatrics, in geriatric nursing, rheumatology, or internal medicine. This is moreover very efficient, because by one single stationary reader the biological data of a large number of patients can be simultaneously detected and processed. Altogether it entails an improvement of medical therapies and results in avoiding unnecessary secondary therapies due to belated diagnoses, respectively. Apart from the feasibility of measurements that can be repeated at will, also the measuring effort itself is simplified. Since measurements, for instance repeated fever measurements, are executed non-invasively as well as without a disturbing loss of mobility through the dermal system, this is of practical importance especially for children and elderly people.

Another and special medical advantage of the dermal diagnostic system of the present invention is the possibility offered by them of a non-invasive detection of individuals that can also be represented in conformity with data protection among a big crowd of people in accordance with a general risk screening. This relates, for instance, within the scope of a prophylaxis against easily transmissible infections, to the early detection of individual travelers as potential risk bearers within mass transportation such as international air traffic. General risks of infection are largely accompanied by increases in the body temperature. Therefore, by early detecting the change of such base parameters an early detection of the transnational ways of spreading and the intensity of newly occurring epidemics is possible. Since the active dermal transponders can be provided with instruments that permit a direct communication with a communication system, especially PCs, portable computers, or mobile phones, it is possible to rapidly transmit and document data that can be exploited for giving an accelerated medical diagnosis. This is an important measure especially within the scope of imminent epidemics for an early prevention thereof. Therefore, it is both considerably helpful in the medical view and cost-saving for further secondary measures.

It is a technical advantage that the dermal diagnostic system of the present invention can be economically and reproducibly manufactured within exact standards on a large scale by usual production systems as well as variably dimensioned in an application-oriented manner. The mechanically flexible components permit coil-to-coil production methods.

Hereinafter a basic example of the present invention is illustrated without intending to technically restrict it to the configuration of this example of an RFID application.

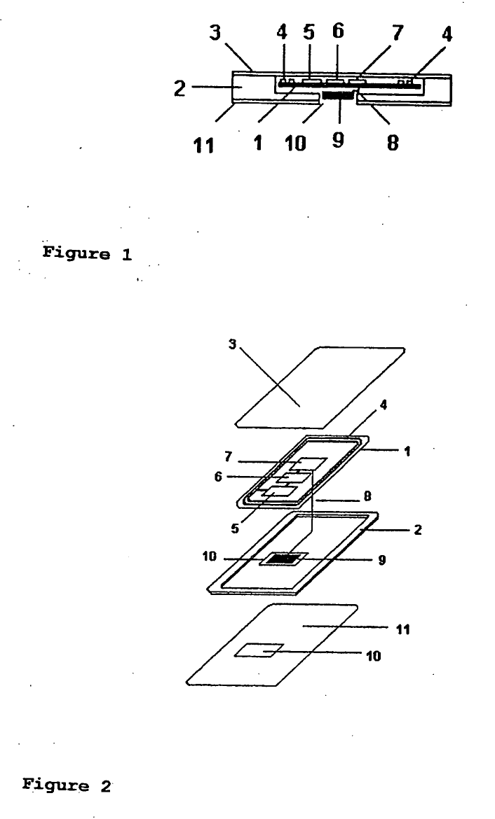

FIG. 1 shows the basic multi-component structure of a dermal system of the present invention including an active transponder in a diagrammatic cross-section. A carrier matrix of a transponder 1 is embedded in this technical embodiment in a top-side recess of a dermal carrier 2 that may include flexible polymeric material in such way that on the whole a planar surface is formed. The components may be connected, for instance, by a one-sided gluing of the lower side of the transponder 1 with a top-side adhesive layer of the carrier 2 or by a lamination enclosing the transponder 1 also on its upper side, wherein then an additional top cover layer 3 is formed, as shown in this example. On the transponder matrix 1 technically connected to the carrier 2 there are provided, in this example, an antenna 4 having a planar circular design, a microchip 5, such as a RFID microchip, an energy source 6, and a specific sensor controller 7, for instance for temperature measurement, although any type of sensor controller can be utilized. In principle, the energy source 6 of the transponder may also be dislocated and possibly may also be separately provided in the carrier 2. The sensor controller 7 of the transponder 1 in this embodiment is electrically connected via a line 8 vertically extending through the dermal carrier 2 with a sensor 9 positioned directly on or above the skin surface, which is made possible by a mechanical recess 10 in the carrier 2. The flexible carrier 2 has on its lower side a cutaneously tolerant adhesive layer 11 that positions and fixes both the carrier 2 itself and the sensor 9 on the skin surface. The entire structure of such a diagnostic system can be manufactured in height dimensions of even below 2 mm by the techniques available at present.

FIG. 2 shows the same diagrammatic structure as shown in picture 1, however in layers as a perspective exploded diagrammatic view seen from the top.

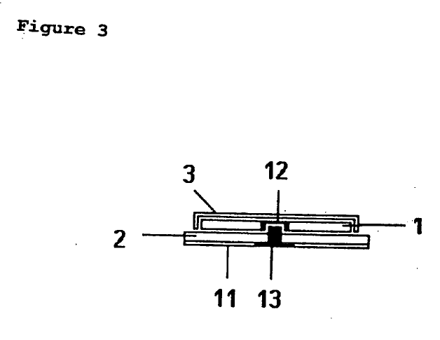

FIG. 3 is another diagrammatic structure, wherein in this case the carrier matrix of the transponder 1 is detachably connected to a skin carrier 2 through a connection system with two connector parts 12, 13, which can be a “snap fit” connection of the snap fastener type. The two complementary parts 12, 13 of the mechanically reversible connecting member, which may be made of metal or a synthetic material, are therefore separately mounted, on the one hand part 12 is mounted in the carrier matrix of the transponder 1 and on the other hand part 13 is mounted in the skin carrier 2. This type of technical configuration of the present invention permits arranging the same transponder 1 in different skin carriers 2 and thus to its reuse several times. The upper cover layer 3 of the transponder 1 in this example is formed as a housing. In the case of a metallic design the snap fit connection 12, 13 can be simultaneously employed also as a transmitter of heat flow from the skin to the temperature sensor.

Further, in the embodiments noted above at least one of an additional electric, magnetic, optical, micromechanical, thermal, chemical or chemical engineering components, or combinations thereof, can be implemented in the carrier 2 or in further carrier materials connected to the carrier 2.

Further, in the embodiment shown in FIG. 3 the detachable arrangement of the transponder 1 and skin carrier 2 is implemented by connections 12, 13, which can be a snap fit connection, but the connection elements 12, 13 can also be implemented chemically by a reversible gluing, magnetically or mechanically by hoop and loop fasteners (velcro), by a flange connection, or by other similar connection.

Further, in the present invention functional parts of the transponder 1 can be arranged separately in the carrier 2 or in further carrier materials connected thereto.

Further, the transponder 1 can include a communication by the antenna 4, microchip 5, etc., of any of optical waves, radio waves, a combination of radio and optical waves, or other communication systems, and may particularly utilize infrared waves.

Further, in the embodiment shown in the figures the sensor 9 can represent plural sensors simultaneously contained in the carrier matrix 2. Further, the sensor 9 can represent a sensor directly integrated in the carrier matrix 2. Further, the sensor 9 can be used for checking at least one of temperature, pulse frequency, blood pressure, moisture, skin resistance, electrical currents produced in the muscles, or current actions of the heart. Further, the sensor 9 can be used for checking chemical parameters of at least one of blood sugar, electrolytes, and lactate.

Further, the antenna 4, microchip 5, etc. of the transponder 1 can be utilized to include electric, magnetic, optical, or micromechanical components configured to directly communicate with a mobile communication device, such as a portable computer, a mobile phone, etc.

The present invention can be used in the fields of human medicine, veterinary medicine, or biology, or in similar fields.

Obviously, numerous modifications and variations of the present invention are possible in light of the above teachings. It is therefore to be understood that within the scope of the appended claims, the present invention may be practiced otherwise than as specifically described herein.

1. A dermal diagnostic system comprising:
an elastic or flexible carrier including an active transponder and at least one sensor, wherein the at least one sensor is in measuring contact with a skin surface or with a subjacent dermal layer, data established by the at least one sensor is supplied to the transponder for external transmission, and wherein an entirety of the dermal diagnostic system is adhesively arranged on the skin surface.

2. A system according to claim 1, further comprising at least one of additional electric, magnetic, optical, micromechanical, thermal, chemical or chemical engineering components, or combinations thereof.

3. A system according to claim 1, wherein at least one of the transponder and the at least one sensor are detachably arranged on the carrier, wherein a detachable arrangement is implemented by complementary connectors on the carrier and the transponder chemically by reversible gluing, magnetically or mechanically by hook and loop fastener (Velcro), snap fastener, or flange connections.

4. A system according to claim 1, wherein functional parts of the transponder are arranged separately in the carrier or in further carrier materials connected therewith.

5. A system according to claim 1, wherein the transponder makes use of at least one of optical waves, radio waves, or a combination of radio and optical waves as signals.

6. A system according to claim 1, wherein the transponder makes use of infrared waves.

7. A system according to claim 1, wherein plural sensors are contained in the carrier.

8. A system according to claim 1, wherein the at least one sensor is directly integrated in the carrier.

9. A system according to claim 1, wherein the at least one sensor is used for checking at least one of temperature, pulse frequency, blood pressure, moisture, skin resistance, electric currents produced in the muscles, or action currents of the heart.

10. A system according to claim 1, wherein the at least one sensor is used for checking chemical parameters of at least one of blood sugar, electrolytes, and lactate.

11. A system according to claim 1, further comprising electric, magnetic, optical, or micromechanical components configured to communicate directly with a mobile communication device.

12. A system according to claim 1, used in the fields of human medicine, veterinary medicine, or biology.

13. A dermal diagnostic system comprising:
elastic or flexible carrier means including active means for transmitting/responding and at least one means for sensing, wherein the at least one means for sensing is in measuring contact with a skin surface or with a subjacent dermal layer, data established by the at least one means for sensing is supplied to the means for transmitting/responding for external transmission, and wherein an entirety of the dermal diagnostic system is adhesively arranged on the skin surface.

14. A system according to claim 13, further comprising at least one of additional electric, magnetic, optical, micromechanical, thermal, chemical or chemical engineering components, or combinations thereof.

15. A system according to claim 13, wherein at least one of the means for transmitting/responding and the at least one means for sensing are detachably arranged on the carrier means, wherein a detachable arrangement is implemented by complementary connectors on the carrier means and the means for transponding/responding chemically by reversible gluing, magnetically or mechanically by hook and loop fastener (Velcro), snap fastener, or flange connections.

16. A system according to claim 13, wherein functional parts of the means for transmitting/responding are arranged separately in the carrier means or in further carrier materials connected therewith.

17. A system according to claim 13, wherein the means for transmitting/responding makes use of at least one of optical waves, radio waves, or a combination of radio and optical waves as signals.

18. A system according to claim 13, wherein the means for transmitting/responding makes use of infrared waves.

19. A system according to claim 13, wherein plural means for sensing are contained in the carrier means.

20. A system according to claim 13, wherein the at least one means for sensing is directly integrated in the carrier means.

21. A system according to claim 13, wherein the at least one means for sensing is used for checking at least one of temperature, pulse frequency, blood pressure, moisture, skin resistance, electric currents produced in the muscles, or action currents of the heart.

22. A system according to claim 13, wherein the at least one means for sensing is used for checking chemical parameters of at least one of blood sugar, electrolytes, and lactate.

23. A system according to claim 13, further comprising electric, magnetic, optical, or micromechanical components configured to communicate directly with a mobile communication device.

24. A system according to claim 13, used in the fields of human medicine, veterinary medicine, or biology.
